(19) United States
(12) Patent Application Publication
(10) Pub. No.: US 2010/0260693 A1
(43) Pub. Date: Oct. 14, 2010

(54) COSMETIC COMPOSITION

(75) Inventors: Tetsuo Kawano, Ashigarakami-gun (JP); Hiroyuki Hirai, Ashigarakami-gun (JP); Kazuhiro Nakamura, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/756,691

(22) Filed: Apr. 8, 2010

(30) Foreign Application Priority Data
Apr. 9, 2009 (JP) 2009-094866

(51) Int. Cl. A61K 8/27; A61Q 17/04; C01G 9/02; B32B 5/16; C07F 3/06

(52) U.S. Cl. 424/59; 423/622; 428/402; 556/9

(57) ABSTRACT

A cosmetic composition contains star-shaped zinc oxide particles each having a star shape, and containing zinc oxide as a main substance thereof.

### BACKGROUND OF THE INVENTION

1. Field of the Invention

The present invention relates to a cosmetic composition, e.g. a foundation, having transparency, UV-shielding properties, and wide-angel scattering properties.

2. Description of the Related Art

An ultraviolet-ray absorbing agent, or ultraviolet-ray scattering agent is generally contained in a cosmetic product, such as a foundation, for the purpose of protecting human skins from ultraviolet rays. As such the ultraviolet-ray absorbing agent, organic compounds have been used, such as benzophenones, paraminobenzoic acids, cinnamates, salicylic acids, dibenzoyl methanes, and benzothiazoles. The ultraviolet-ray absorbing agent has a restriction for its amount formulated to cosmetics, and it is also known that the ultraviolet-ray absorbing agent has a problem in the safety, such as irritation to skins. Furthermore, as the wavelength of the ultraviolet ray to which the ultraviolet-ray absorbing agent shows its maximum absorption is different depending on the structure of the ultraviolet-ray absorbing agent, it is necessary to combine a few types of the ultraviolet-ray absorbing agents, or to combine the ultraviolet-ray absorbing agent with the ultraviolet-ray scattering agent. In addition, the ultraviolet-ray absorbing agent which has a low solubility to water or oils may cause precipitation of crystals thereof, and thus may be difficult to be formulated into cosmetics.

As the ultraviolet-ray scattering agent, inorganic pigments have been used, such as titanium oxides, and zinc oxides. These inorganic pigments are chemically and physically stable, highly safe to use, and can physically shield ultraviolet rays so that skins can be protected from ultraviolet rays of a wide wavelength range. Moreover, they exhibit an effect of an ultraviolet-ray absorbing agent because of absorption due to an interband transition, in which an electron is excited from a valence band to a conduction band.

However, the conventional foundations containing these inorganic pigments cannot sufficiently satisfy, for example, a masking effect and a UV-blocking effect only by adding the inorganic pigments, and thus it has been necessary to formulate both a material for providing the masking effect (wide-angle scattering) and a material for providing the UV-blocking effect. Moreover, the conventional foundations containing the inorganic pigments have problems, such as thickly adhered feeling, excessively white finish, and lowering in the chroma of the foundation.

To solve the aforementioned problem, it has been proposed, for example in Japanese Patent Application Laid-Open (JP-A) No. 2006-76798, titanium oxide particles, each of which has a plurality of radially extended portions and has ridges at about the middle part of the extended portions in the thickness direction thereof so that each particle has a shape of a star on the whole, and a cosmetic containing such titanium oxide particles.

Moreover, it has been announced in the website (URL:http://release.nikkei.co.jp/detail.cfm?relID=209775&lindID=4, released on Jan. 19, 2009) that Kanebo Cosmetics Inc. is going to release, on Mar. 16, 2009, a whitening base make-up brand “Kanebo Brawn Seal Superior” which uses the star-shaped titanium oxide particles developed by SUMITOMO OSAKA CEMENT Co., Ltd. (see Technical Report 2007 of SUMITOMO OSAKA CEMENT Co., Ltd., pp. 28-31, the first published date of Dec. 8, 2006). In this article, it is disclosed that by adding the star-shaped titanium oxide particles capable of uniformly scattering light in a wide range, the effect of light would uniformly cover skin, thus resulting in the finish having transparency.

However, in the aforementioned prior part documents, it is necessary to use a titanium metal salt, e.g. titanalkoxide or titanium tetrachloride, as a raw material to obtain titanium oxide particles (the titanium oxide particles each having a star shape as the whole). Since titanalkoxide or titanium tetrachloride is expensive, and also requires caution for handling in the atmosphere containing moisture, there is a problem that the cost of the finally obtained titanium oxide particles is increased influenced by the cost incurred by the materials or the complication of the production process. In this case, it is more desirable that the process producing the star-shaped titanium oxide particles more simply and inexpensive, but the aforementioned prior art documents have not disclose or suggest such the process. Moreover, it is also presumed that the particles having the composition on merit of the cost can be used instead of the star-shaped titanium oxide particles, but the aforementioned prior art documents have not disclose or suggest such the particles. It is a current situation such that the prior art documents have not disclose or suggest the use of star-shaped zinc oxide particles, which are clearly different from the star-shaped titanium oxide particles in terms of their materials, physical properties and structures, to cosmetic products such as foundations.

### BRIEF SUMMARY OF THE INVENTION

An object of the present invention is to provide a cosmetic composition having transparency, UV-blocking properties to the ultraviolet rays in the UV-A range (320 nm to 400 nm), and wide-angel scattering properties capable of uniformly scatter light to a wide area at low cost.

The means for solving the aforementioned problem are as follows.

<1> A cosmetic composition, containing:

star-shaped zinc oxide particles each having a star shape, and containing zinc oxide as a main substance thereof.

<2> The cosmetic composition according to <1>, wherein the star-shaped zinc oxide particles are formed by accumulating particles having a diameter of 1 nm to 100 nm so that crystallographic orientations thereof are aligned in the same direction, and allowing the particles to grow in a branched manner, and wherein the star-shaped zinc oxide particles have an average particle diameter of 1 μm to 3 μm.
<3> The cosmetic composition according to <1>, wherein an amount of the star-shaped zinc oxide particles is 0.1% by mass to 90% by mass with respect to the total amount of the cosmetic composition.
<4> The cosmetic composition according to <1>, wherein the star-shaped zinc oxide particles are subjected to a surface treatment with a surface-treating agent.
<5> The cosmetic composition according to <4>, wherein the surface-treating agent is a silane-coupling agent.
<6> The cosmetic composition according to <1>, wherein the cosmetic composition is a foundation.

According to the present invention, the conventional problems in the art can be solved, and a cosmetic composition having transparency, UV-blocking properties to the ultraviolet rays in the UV-A region (320 nm to 400 nm), and wide-angel scattering properties capable of uniformly scatter light to a wide area can be provided at low cost.

### BRIEF DESCRIPTION OF THE DRAWINGS

FIG. 1A is a TEM photograph of the star-shaped zinc oxide particles used in Example 1.

FIG. 1B is an enlarged TEM photograph of FIG. 1A.

FIG. 1C is a SEM photograph of the star-shaped zinc oxide particles used in Example 1.

FIG. 1D is an enlarged SEM photograph of FIG. 1C.

FIG. 2 is a TEM photograph of the commercially available zinc oxide particles used in Comparative Example 2.

FIG. 3 shows diffuse reflectance spectrums of samples respectively formed by applying to a glass substrate an aqueous dispersion liquid of the star-shaped zinc oxide particles used in Example 1 and an aqueous dispersion liquid of the commercially available zinc oxide particles used in Comparative Example 2, and then drying.

FIG. 4 is a graph showing the results of the star-shaped zinc oxide particles used in Example 1 and the titanium oxide particles used in Comparative Example 1, measured by a goniometer device.

### DETAILED DESCRIPTION OF THE INVENTION

The cosmetic composition of the present invention contains at least star-shaped zinc oxide particles, and may further contain other substances, if necessary.

### <Star-Shaped Zinc Oxide Particles>

The star-shaped zinc oxide particles each have a star shape, and contain zinc oxide as a main substance.

Here, the star-shaped zinc oxide particles are described as containing zinc oxide as a main substance, because, other than the case they are formed only of zinc oxide, there are cases where they contain zinc oxide to which a small amount of an element such as Mg is doped, or contain a mixture of zinc oxide and other inorganic compounds. Specifically, the amount of zinc oxide contained in the star-shaped zinc oxide particles is preferably 50% by mass or more, more preferably 90% by mass or more, yet more preferably 100% by mass.

Zinc oxide (ZnO), which is a main substance of the star-shaped zinc oxide particles, is excellent for shielding the ultraviolet rays of UV-A range (320 nm to 400 nm) as it has an adsorption peak at around the wavelength of 380 nm, is inexpensive and is excellent in transparency. On the other hand, titanium oxide is excellent for shielding the ultraviolet rays of UV-B range (290 nm to 320 nm) as it has an adsorption peak at around the wavelength of 300 nm.

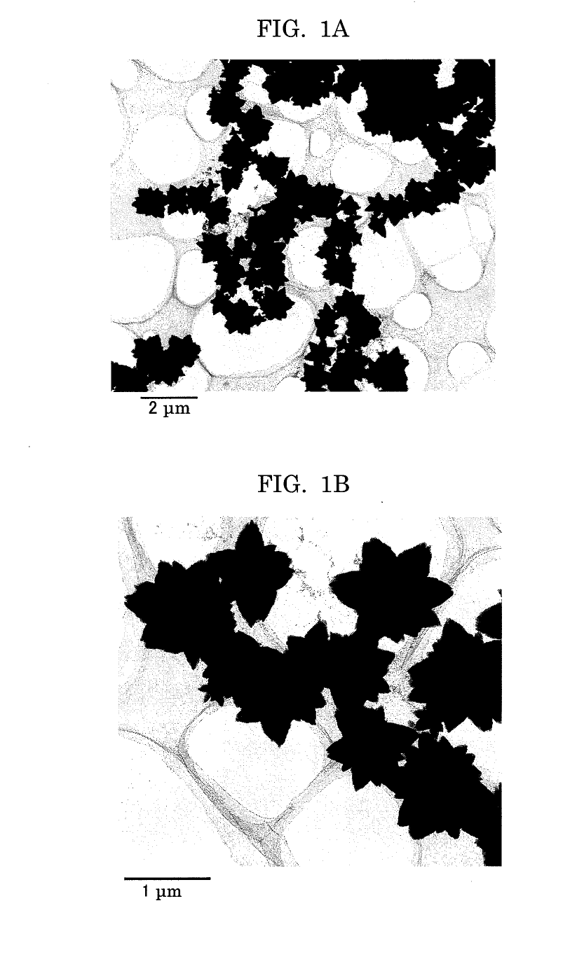

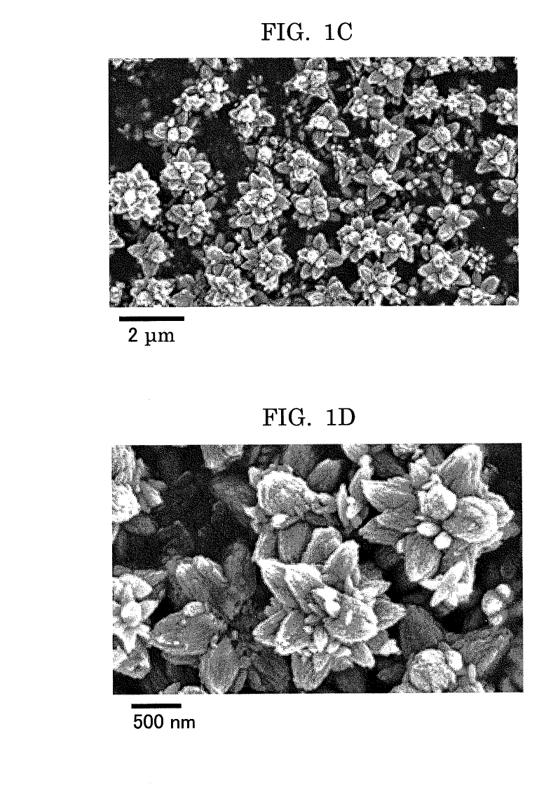

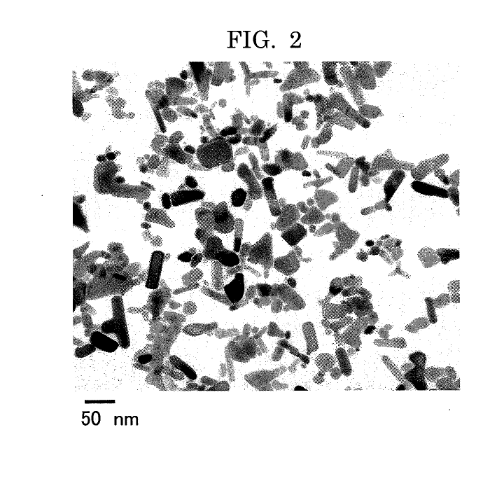

As shown in FIGS. 1A to 1D, the star shape refers to a shape formed as a result that particles having a diameter of 1 nm to 100 nm (preferably 25 nm to 40 nm) are accumulated so that crystallographic orientations thereof are aligned in the same direction, and grown in a branched manner so as to form a plurality of projections, each of which radially extended from a core located at substantially the center of the projection in its longitudinal direction, and forming a star as a whole. The number of the projections is at least 6, preferably 6 to 10.

On the other hand, as shown in the images liked with the website (URL:http://release.nikkei.co.jp/detail.cfm?relID=209775&lindID=4, released on Jan. 19, 2009) and Technical Report 2007 of SUMITOMO OSAKA CEMENT Co., Ltd., pp. 28-31, the first published date of Dec. 8, 2006, each of the star-shaped titanium oxide particles disclosed in JP-A No. 2006-76798 is substantially in the shape of a plate containing six extended portions each being of a monocrystal, and is a twin crystal as a whole.

Accordingly, the star-shaped zinc oxide particles are different from the star-shaped titanium oxide particles in terms of their materials, physical properties, structure and shapes.

Whether or not the zinc oxide particles have the star shapes can be determined by observing the zinc oxide particles under a scanning electron microscope (SEM) or a transmittance electron microscope (TEM).

The average particle diameter of the star-shaped zinc oxide particles is preferably 1 μm to 3 μm. When the average particle diameter thereof is less than 1 μm, the aggregations of the star-shaped zinc oxide particles may occur significantly. When the average particle diameter thereof is more than 3 μm, it may be necessary to adapt the process, such as extending the reaction time in the reaction process.

Note that, the average particle diameter can be measured, for example, by observing the star-shaped zinc oxide particles under a scanning electron microscope (SEM) or a transmission electron microscope (TEM) and measuring the obtained image by a caliper, or measuring using an image analyzer.

Specifically, it can be measured by placing the star-shaped zinc oxide particles, determining the longest length of the lines each of which starts from the edge of one projection (branch) to the edge of the opposed projection (branch) via the center portion of the particle, as a particle diameter, measuring the particle diameter on randomly selected 50 star-shaped zinc oxide particles, and then obtaining and determining the average value thereof as the average particle diameter.

The amount of the star-shaped zinc oxide particles is preferably 0.1% by mass to 90% by mass, more preferably 0.2% by mass to 70% by mass, yet more preferably 0.3% by mass to 50% by mass with respect to the total amount of the cosmetic composition. When the amount thereof is less than 0.1% by mass, it may be not able to obtain the cosmetic composition having the required shielding properties of ultraviolet rays. When the amount thereof is more than 90% by mass, there may be problems in handling or coating to skins.

Note that, the cosmetic composition of the present invention may contain, other than the star-shaped zinc oxide particles, metal oxide particles, e.g. titanium oxide and selenium oxide, and/or organic ultraviolet-ray absorbing agents, as the ultraviolet-ray shielding agent.

The star-shaped zinc oxide particles for use in the present invention can be produced by the production method of star-shaped zinc oxide particles, which will be explained hereinafter.

### <Production Method of Star-Shaped Zinc Oxide Particles>

The production method of star-shaped zinc oxide particles contains a heating step and a diluting step, and may further contain other steps, if necessary. The order of the heating step and the diluting step may not be a matter, whichever can be performed first, and these steps can also be performed at the same time.

### <<Heating Step>>

The heating step is heating a solution containing tetrahydroxozincate (II) ions [Zn(OH)₄]².

### —SOlution Containing Tetrahydroxozincate (II) Ions [Zn(OH)4]²⁻—

The solution containing tetrahydroxozincate (II) ions [Zn(OH)4]²⁻ preferably contains zinc ions (Zn²⁺) and hydroxide ions (OH⁻) and preferably has the pH value of 12.5 to 14.0. When the pH value thereof is less than 12.5, the yield of the star-shaped zinc oxide particles may decreases. When the pH value thereof is more than 14.0, the precipitation itself may not occur as the solubility of zinc oxide is high.

The pH value is the value measured at the temperature of 25° C. using a pH meter.

As the solution containing tetrahydroxozincate (II) ions [Zn(OH)4]²⁻, for example, a mixture of an aqueous solution of an alkali compound and an aqueous solution of a zinc compound may be used.

The alkali compound is suitably selected depending on the intended purpose without any restriction. Examples thereof include: hydroxides of alkali metals such as sodium hydroxide, and potassium hydroxide; carbonates of alkali metals such as sodium carbonate and potassium carbonate; sodium acetate; ammonium compounds such as ammonium gas, ammonia water, and ammonium hydroxide; alkyl amines such as ethyl amine, propyl amine, butyl amine, and ethylene diamine; and alkanol amines such as monoethanol amine, diethanol amine, triethanol amine, N,N-dimethylethanol amine, monopropanol amine, dipropanol amine, and tripropanol amine. These may be used independently, or in combination. Among them, sodium hydroxide is particularly preferable.

The concentration of the alkali compound in the aqueous solution of the alkali compound is suitably adjusted depending on the intended purpose without any restriction.

The zinc compound is suitably selected depending on the intended purpose without any restriction. Examples thereof include zinc hydroxide, zinc sulfate, zinc nitrate, zinc chloride, and zinc acetate. Among them, zinc sulfate is particularly preferable.

The concentration of the zinc compound in the aqueous solution of the zinc compound is suitably selected depending on the intended purpose without any restriction.

The aqueous solution of the alkali compound and the aqueous solution of the zinc compound are mixed, and optionally stirred. As a result of this, followed by the reaction, tetrahydroxozincate (II) ions [Zn(OH)₄]²⁻ are generated as an intermediate product.

It is preferred that the aqueous solution of the alkali compound and the aqueous solution of the zinc compound be mixed at the room temperature (25° C.) or the temperature lower than the room temperature. The aqueous solution of the zinc compound may be added to the aqueous solution of the alkali compound once or gradually in a few times. As in the manner mentioned above, a clear solution containing tetrahydroxozincate (II) ions [Zn(OH)4]²⁻ is obtained.

The temperature lower than the room temperature is preferably the temperature in the range of 0° C. to 20° C.

For the aforementioned stirring, the commonly used mixing and stirring devices can be used. For example, the stirring can be carried out by using a stirrer having a stirring blade, and the like.

### —Heating—

Next, the solution containing tetrahydroxozincate (II) ions [Zn(OH)₄]²⁻ is heated.

The heating temperature of the solution containing tetrahydroxozincate (II) ions [Zn(OH)₄]²⁻ is preferably 40° C. or higher, more preferably 45° C. or higher.

The upper limit of the heating temperature is different depending on the solvent for use, and cannot be determined unconditionally. In the case where water is used as the solvent, the upper limit of the heating temperature is preferably lower than 100° C., more preferably 90° C. or lower.

When the heating temperature is lower than 40° C., the progress of the reaction may be slow. When the heating temperature is 100° C. or higher, the reaction cannot be carried out using water as the solvent under the atmospheric pressure, and thus a reaction vessel such as an autoclave may be required.

It is preferred that the heating temperature be maintained until the reaction is completed.

Moreover, the method for heating is suitably selected depending on the intended purpose without any restriction. Suitable examples thereof include a method in which the solution containing tetrahydroxozincate (II) ions [Zn(OH)₄]²⁻ is added to a solvent which has been heated at a predetermined temperature in advance. In accordance with this method, heating and diluting the solution containing tetrahydroxozincate (II) ions [Zn(OH)₄]²⁻ are performed at the same time.

### <<Dilution Step>>

The dilution step is a step for adding a solvent to the solution containing tetrahydroxozincate (II) ions [Zn(OH)₄]²⁻ in the middle of the reaction so as to dilute the solution, to thereby control the concentration of the zinc ion (Zn²⁺) after the dilution of the solution to be less than 0.01 M.

### —Solvent—

Examples of the solvent include water, methanol, ethanol, isopropyl alcohol, and the like. Among them, water is particularly preferable. The solvent is added to the solution containing tetrahydroxozincate (II) ions [Zn(OH)₄]²⁻ in the middle of the reaction so as to dilute the solution. By diluting the solution by adding the solvent in the middle of the reaction in the aforementioned manner, the pH value of the reaction liquid and the concentration of zinc ion thereof can be controlled and the star-shaped zinc oxide particles can be formed.

The aforementioned timing, i.e. “in the middle of the reaction,” is suitably selected depending on the intended purpose without any restriction, provided that it is sometime between the beginning of the reaction and the termination of the reaction, and may be before, or after, or at the same time when the solution containing tetrahydroxozincate (II) ions [Zn(OH)₄]²⁻ is heated.

The amount of the solvent to be added is the amount so that the concentration of a zinc ion (Zn²⁺) is to be 0.008 M or less after the dilution of the solution, and preferably the amount so that the concentration thereof is to be 0.005 M or less. Note that, the lower limit of the concentration of a zinc ion (Zn²⁺) after the dilution of the solution is suitably selected depending on the intended purpose without any restriction, is theoretically preferably 0.008 M. When the concentration of a zinc ion (Zn²⁺) after the dilution of the solution is more than 0.008 M, the yield of the star-shaped zinc oxide particles may be decreased unless the reaction conditions are optimized.

When the concentration of a zinc ion (Zn²⁺) after the dilution of the solution is 0.006 M to 0.008 M, the star-shaped zinc oxide particles are obtained, but zinc hydroxide is also generated. Therefore, the star-shaped zinc oxide particles coexist with zinc hydroxide. Such the concentration of the zinc ion after the dilution of the solution that only the star-shaped zinc oxide particles are formed is 0.005 M or less.

The crystal phase of the obtained particles (whether they are consisted of zinc hydroxide, zinc oxide, or a mixture thereof) can be determined, for example, by X-ray diffraction (XRD).

The method for adding the solvent (the dilution method with the solvent) is suitably selected depending on the intended purpose without any restriction. Since it is not hardly influenced by the adding speed, the entire amount of the solution containing tetrahydroxozincate (II) ions [Zn(OH)₄]²⁻ may be added to the solvent once, or the solution containing tetrahydroxozincate (II) ions [Zn(OH)₄]²⁻ may be gradually added to the solvent in a few times.

The reaction time cannot be determined unconditionally as it defers depending on the reaction temperature (heating temperature) and the like. For example, in the case where the reaction temperature is 60° C., the reaction time is preferably 2 hours or longer.

Moreover, the pH value of the solution after the completion of the reaction is preferably 11 to 13. When the pH value thereof is less than 11, ZnO may not be present as a monocrystal. When the pH value thereof is more than 13, the yield of the star-shaped zinc oxide particles may decrease.

The aforementioned pH value is a value measured at 25° C. using a pH meter.

The production method of the star-shaped zinc oxide particles is, for example, the following method. Namely, to a solution which has been prepared by adding a NaOH solution to pure water, a ZnSO₄ solution is mixed at room temperature to thereby prepare a clear solution.

Then, the clear solution is added to water, which is a solvent and has been heated to 60° C. in advance, and the mixture is allowed to react at 60° C. for 2 hours, to thereby obtain star-shaped zinc oxide particles.

According to the production method of the star-shaped zinc oxide particles, star-shaped zinc oxide particles can be efficiently produced with a simple process, without any special devices.

### —Surface Treatment—

It is preferred that the star-shaped zinc oxide for used in the present invention be surface-treated with a surface-treating agent, as such the star-shaped zinc oxide particles can be uniformly dispersed in the cosmetic composition.

The surface-treating agent is suitably selected depending on the intended purpose without any restriction. Examples thereof include a silane-coupling agent, a silanizing agent, a silane-coupling agent containing a fluoroalkyl group, an organic titanate-based coupling agent, an aluminum-based coupling agent, silicone oil, and modified silicone oil. Among them, the silane-coupling agent is particularly preferable.

Examples of the silane-coupling agent include methyltrimethoxy silane, methyltriethoxy silane, octyltrimethoxy silane, and octyltriethoxy silane.

The cosmetic composition of the present invention is suitably modified depending on the intended purpose without any restriction, provided that it contains the star-shaped zinc oxide particles. Examples of the cosmetic composition include a toner, a moisturizer, a cream, ointment, a foundation, a lip balm, a lip stick, a mascara, an eye shadow, an eye brow pencil or marker, and a nail polish. Among them, the foundation is particularly preferable.

The foundation is a cosmetic product, which is applied onto an entire face so that stains, freckles, fine wrinkles, pores and downy hairs present on the face are covered, controlling the tone of the face color even.

The foundation is available in the form of a liquid, a powder, a cream, a stick (a concealer), and the like. As for the examples of the foundation, a liquid foundation and a powder foundation will be explained hereinafter.

### <Liquid Foundation>

The liquid foundation contains the star-shaped zinc oxide particles, an oil substance, an aqueous substance, an emulsifier, and a coloring agent, and may further contain other substances, as necessary.

The substances other than the star-shaped zinc oxide particles are suitably selected from commercially available raw materials for cosmetics without any restriction. Note that, the liquid foundation include a foundation in the form of foam, and a foundation in the form of a gel.

### —Oil Substance—

The oil substance is suitably selected depending on the intended purpose without any restriction. Examples thereof for use in the conventional cosmetics include a volatile oil solution, a nonvolatile oil solution, a solvent, and a resin. These may be in the form of a fluid, a paste, or a solid at room temperature. Specific examples of the oil substance include: higher alcohol such as cetyl alcohol, isostearyl alcohol, behenyl alcohol, octyl dodecanol, and cholesterol; fatty acids such as isostearic acid, stearic acid, and oleic acid; esters such as myristyl myristate, hexyl laurate, decyl oleate, isocetyl myristate, cetyl octanoate, isononyl isononanoate, isododecyl isononanoate, octyldodecyl oleate, and octyl oxystearate; hydrocarbons such as liquid paraffin, vaseline, and squalene; wax such as lanoline, reduced lanoline, and carnauba wax; oils and fats, such as mink oil, cocoa butter, shea butter, camellia oil, sesame oil, castor oil, and olive oil; fluorine-based oil solutions such as perfluoropolyether; and ethylene/α-olefin co-oligomer. These may be used independently or in combination.

Moreover, examples of other forms of the oil substance include: silicone compounds such as dimethyl polysiloxane, methylhydrogen polysiloxane, methylphenyl polysiloxane, polyether-modified organopolysiloxane, alkyl-modified organipolysiloxane, organopolysiloxane having a modified terminal group(s), polyglycerol-modified silicone, polyglycerol-modified silicone, amono-modified organopolysiloxane, cyclic silicone tetra- to hexamer, silicone gel, acrylic silicone, and trimethylsiloxy silicic acid; and fluorine compounds such as perfluoropolyether. These may be used independently or in combination.

### —Aqueous Substance—

Examples of the aqueous substance include: polyhydric alcohols such as glycerin, sobitol, mannitol, propylene glycol, polyethylene glycol, and raffinose; and purified water. These may be used independently or in combination.

### —Emulsifier—

As for the emulsifier, for example, an anionic surfactant, a cationic surfactant, a nonionic surfactant, and an amphoteric surfactant can be used. Among them, the anionic surfactant and the nonionic surfactant are particularly preferable. Examples of the emulsifier include alkyl sulfate, higher fatty acid soap, glycerin fatty acid ester, propylene fatty acid ester, sorbitan fatty acid ester, polyoxyethylene alkylether, polyoxyethylene sorbitan fatty acid ester, and polyether-modified silicone. These may be used independently or in combination.

### —Coloring Agent—

The coloring agent is suitably selected depending on the intended purpose without any restriction. Examples thereof include: organic coloring agents such as Red No. 104 aluminum lake, Red No. 102 aluminum late, Red No. 226, Red No. 201, Red No. 202, Blue No. 404, Blue No. 1 aluminum lake, Yellow No. 4 aluminum lake, Yellow No. 5 aluminum lake, and Yellow No. 203 barium lake; color pigments such as yellow iron oxide, iron red, black iron oxide, chromium oxide, carbon oxide, ultramarine blue, and iron blue; extender pigments such as talc, mica, sericite, kaolin, and tabular barium sulfate; peal pigments such as a titanium-mica composite material; metal salts such as barium sulfate, calcium carbonate, magnesium carbonate, aluminum silicate, and magnesium silicate; inorganic powders such as silica, spherical silica, and alumina; and polymers such as bentonite, smectite, boron nitride, a nylon powder, a silk powder, an urethane powder, a Teflon (registered trade mark) powder, a silicone powder, a methyl polymethacrylate powder, a cellulose powder, a silicone elastomer spherical powder, and a polyethylene powder. These may be used independently or in combination.

The shapes (e.g. sphere, rod, needle, plate, irregular, scale, spindle, and the like) of these powder are suitably selected depending on the intended purpose without any restriction. The size of the powder is preferably 5 nm to 100 μm, more preferably 10 nm to 25 μm. Among the coloring agents listed above, inorganic coloring agents such as iron oxide, titanium oxide, talc, mica, and sericite are preferably used.

### —Other Substances—

The aforementioned other substances are suitably selected depending on the intended purpose without any restriction. Examples thereof include vitamins, amino acids, sterilizers, antiinflammatory agents, animal or plant extracts and derivatives thereof, antiseptic agents, pH regulators, antioxidants, chelating agents, moisturizing agents, and fragrance materials.

The vitamins are suitably selected depending on the intended purpose without any restriction. Examples thereof include vitamin A, vitamin B complex, vitamin C, vitamin D, vitamin E, vitamin F, vitamin K, vitamin P, vitamin U, carnitine, ferulic acid, γ-orizanol, α-lipoic acid, orotic acid, and derivatives thereof. These may be used independently or in combination.

The amino acids are suitably selected depending on the intended purpose without any restriction. Examples thereof include glycine, alanine, valine, leucine, isoleucine, pehnylalanine, tryptophan, cystine, cysteine, methionine, proline, hydroxyproline, aspartic acid, glutamic acid, arginine, histidine, lysine, and derivetives thereof. These may be used independently or in combination.

The sterilizers are suitably selected depending on the intended purpose without any restriction. Examples thereof include isopropylmethylphenol, and triclosan. These may be used independently or in combination.

The antiinflammatory agents are suitably selected depending on the intended purpose without any restriction. Examples thereof include dipotassium glycyrrhetinate, and stearyl glycyrrhetinate. These may be used independently or in combination.

The animal or plant extracts and derivatives thereof are suitably selected depending on the intended purpose without any restriction. For example, natural extracts, such as plant extracts, seaweed extracts, and herbal medicines are preferable. Specific examples thereof include Angelica keiskei extract, avocado extract, sweet Hydrangea leaf extract, Althaea extract, Arnica extract, Aloe barbadensis leaf extract, Prunus armeniaca (appricot) fruit extract, Prunus armeniaca (appricot) kernel extract, ginkgo extract, turmeric extract, oolong tea extract, rose fruit extract, Echinacea angustifolia leaf extract, Scutellaria root extract, cork tree bark extract, barley extract, St. John's wort extract, Lamium album extract, watercress extract, orange extract, dried sea products, hydrolyzed elastin, hydrolyzed wheat powder, hydrolyzed silk, chamomile extract, Daucus carota sativa (carrot) root extract, Arthemisia capillaries extract, hibiscus extract, kiwi fruit extract, cinchona bark extract, cucumber extract, quanosine, Gardenia florida extract, Bambuseae sasa extract, Sophora angustifolic extract, walnut extract, grapefruit extract, Clematis vitalba extract, chlorella extract, mulberry extract, Gentiana lutea root extract, tea extract, yeast extract, burdock extract, rice bran fermented extract, rice germ oil, comfrey extract, collagen, Garcinia combogia extract, Asarim sieboldi root extract, Saikokaryukotsuboreito extract, umbilical extract, Salvia officinalis leaf extract, Saponaria officinalis extract, bamboo sasa extract, hawthorm berry extract, Zanthoxylum extract, Lentinus edodes extract, Rehmannia root extract, Lithospermum root extract, perilla extract, linden extract, meadowsweet extract, peony root extract, Acous calamus root extract, white birch extract, Equisetum arvense extract, ivy extract, hawthorn berry extract, elder flower extract, yarrow extract, pepper mint extract, sage extract, mallow extract, Cnidium officinale root extract, Swertia japonica extract, soy extract, jujube extract, thyme extract, Imperate cylindrica root extract, Citrus unshiu peel extract, Angelica acutiloba root extract, Calendula officinalis flower extract, peach kernel extract, wild orange peel extract, Houttunia cordata extract, tomato extract, fermented soybeans extract, carrot extract, garlic extract, Rose canina fruit extract, Ophiopogon tuber extract, lotus extract, parsley extract, beeswax, Parietaria judaica extract, Isodonis japonicus extract, bisabolol, Tussilago farfara extract, butterbur root extract, Poria cocos extract, Ruscus aculeates root extract, grape extract, propolis, Luffa cylindrica extract, safflower extract, peppermint extract, Tilia miqueliana extract, peony extract, hops extract, pine extract, skunk cabbage extract, Sapindus mukurossi extract, peach extract, Centaurea cyanus flower extract, eucalyptus extract, Saxifraga sarmentosa extract, Citrus junos fruit extract, coix seed extract, Artemisia mongolia leaf extract, lavender extract, lettuce extract, lemon extract, Astragalus sinicus extract, rose extract, Anthemis nobilis flower extract, and royal jelly extract. These may be used independently or in combination.

The antiseptic agents are suitably selected depending on the intended purpose without any restriction. Examples thereof include aminoethyl sulfonate, benzoic acid, sodium benzoate, benzyl benzoate, benzoin, liquefied phenol, ethanol, edetate sodium, cetylprridinium chloride, benzalkonium chloride, benzethonium chloride, sodium sulfate, agar, dl-camphor, Powerful Sanplezer N (product name) (manufactured by San-Ei Gen F.F.I., Inc.), citric acid, sodium citrate, chlorocresol, chlorobutanol, gentisic acid ethanolamide, salicylic acid, sodium salicylate, dibutylhydroxytoluene, 2,6-di-t-butyl-4-methylphenol, Seisept (product name)(manufactured by Seiwa Kasei Co., Ltd.), sorbic acid, potassium sorbate, nitrogen, thymol, dehydroacetic acid, sodium dehydroacetate, 2-naphthol, hinokitiol, sucrose, honey, isobutyl paraoxybenzoate, isopropyl paraoxybenzoate, ethyl paraoxybenzoate, butyl paraoxybenzoate, propyl paraoxybenzoate, methyl paraoxybenzoate, paraformaldehyde, phenylethyl alcohol, phenol, Proxyel GXL (product name) (manufactured by AstraZeneca K.K.), benzyl alcohol, boric acid, borax, d-borneol, l-menthol, eucalyptus oil, oxyquinoline sulfate, isopropylmethylphenol, undecylenic acid monoethanolamide, distearyldimethylammonium chloride, stearyldimethylbenzylammonium chloride, stearyltrimethylammonium chloride, alkyl diaminoethylglycine hydrochloride liquid, chlorhexidine hydrochloride (gluconate), orthophenylphenol, cresol, chloramines T, chloroxylenol, chlorphenesin, alkyl isoqunolinium bromide liquid, domiphen bromide, thianthol, trichlorocarbanilide, parachlorophenol, halocarban, 2-(2-hydroxy-5-methylphenyl)benzotriazole, hexachlorophene, resorcin, and phenoxy ethanol. These may be used independently or in combination.

The pH regulators are suitably selected depending on the intended purpose without any restriction. Examples thereof include acid, base, and salts of the base. These pH regulators may be used independently or in combination. Among them, triethanol amine, and triisopropanol amine are preferable.

The antioxidants are suitably selected depending on the intended purpose without any restriction. Examples thereof include dibutylhydroxytoluene, butylhydroxyanisole, and ascorbic acid. These may be used independently or in combination.

The chelating agents are suitably selected depending on the intended purpose without any restriction. Examples thereof include disodium edetate, ethylenediamine tetraacetate, hexametaphosphate, and gluconic acid. These may be used independently or in combination.

Examples of the moisturizing agents include serine, glycine, threonine, alanine, collagen, hydrolyzed collagen, hydronectin, fibronectin, keratin, elastin, royal jelly, chondroitin sulfuric acid heparin, glycerophospholipid, glyceroglycolipid, sphingophospholipid, sphingoglycolipid, linoleic acid and esters thereof, eicosapentaenoic acid and esters thereof, pectin, bifidobacteria fermented products, lactic acid fermented products, yeast extract, culture medium of Ganoderma lucidum mycelia and extracts thereof, wheat germ oil, avocado oil, rice germ oil, jojoba oil, soy phospholipid, γ-orizanol, Althae officinalis extract, coix seed extract, Rehmannia root extract, jujube extract, seaweed extract, Aloe arborescens leaf extract, burdock extract, rosemary extract, Arnica extract, and wheat bran. These may be used independently or in combination.

Examples of the flagrance materials include menthol, carvone, eugenol, anethole, menthe oil, spearmint oil, peppermint oil, eucalyptus oil, and anise oil. These may be used independently or in combination.

The liquid foundation is either of W/O or of O/W, depending on the way of using the emulsifier, but either of them is acceptable in the present invention.

### <Powder Foundation>

The powder foundation is a fine powder composition formed by pulverizing the star-shaped zinc oxide particles, an oil substance, a coloring agent, and optionally selected additives, and providing the mixture with friction. It is preferred that the powder foundation be prepared so that the average particle diameter thereof be adjusted to 10 μm or less, more preferably approximately 1 μm, to thereby provide the powder foundation with characteristics such that it spreads on and is fitted to skins. The average particle diameter of the powder foundation can be measured, for example, by using an image of the powder obtained by a scanning electron microscope (SEM) or a transmission electron microscope (TEM), and determining an equivalent circle diameter as the particle diameter thereof.

The materials and formulation of the powder foundation are suitably selected depending on the intended purpose without any restriction, and the oil substance, the coloring agent, and the optionally selected additives for used in the powder foundation can be those used in the liquid foundation mentioned earlier.

### EXAMPLES

Examples of the present invention will be explained hereinafter, but they shall not be construed as limiting the scope of the invention.

In the following examples and comparative examples, a pH value, average particle diameter, and X-ray diffraction (XRD) were measured in the following manners.

<Measurement of pH>

The pH value was measured at 25° C. using a portable pH meter HM-21P, manufactured by DKK-TOA Corporation.

### <Measurement of Average Particle Diameter>

The average particle diameter was obtained by measuring sizes of 50 randomly selected particles on the image obtained by a scanning electron microscope (SEM) or a transmission electron microscope (TEM), and calculating the average value therefrom.

In the case where the particles are the star-shaped zinc oxide particles, it was measured by placing the star-shaped zinc oxide particles, determining the longest length of the lines each of which starts from the edge of one projection (branch) to the edge of the opposed projection (branch) via the center portion of the particle, as a particle diameter, measuring the particle diameter on randomly selected 50 star-shaped zinc oxide particles, and then obtaining and determining the average value thereof as the average particle diameter.

In the case of particles having other shapes (polygon, irregular shapes, and sphere), a circle equivalent diameter was determined as a particle diameter.

### <X-ray Diffraction (XRD) Analysis>

The obtained particles were analyzed in accordance with X-ray diffraction (XRD) using RINT 1500 (X-ray source: CuKa ray, wavelength: 1.5418 Å) manufactured by Rigaku Corporation, so as to determine the crystal phase thereof (whether thay are zinc hydroxide, zinc oxide, or a mixture thereof).

### Synthesis Example 1

### Synthesis of Star-shaped Zinc Oxide Particles

Into a beaker (made of plastic), a solution in which 12.5 mL of a 4.0M NaOH solution had been added to 25.0 mL of pure water was pored, then 12.5 mL of a 0.2M ZnSO₄ solution was added thereto. The mixture (50 mL in total) was mixed at room temperature (25° C.). This clear solution is referred to a solution A. The solution A had a pH value of 13.5.

Next, into 450 mL of water which had been heated to 60° C. in advance, 50 mL of the solution A was added, and the mixture was allowed to react at 60° C. for 2 hours. The solution had the pH value of 12.3 after the reaction. The obtained particles were sufficiently washed by decantation or filtration, and then dried. Finally, the particles were subjected to a surface treatment with octyltriethoxysilane. In the manner mentioned above, zinc oxide particles were obtained.

Note that, the stirring was performed during the reaction by adjusting the stirring condition (rotation number) so that the solution had a state where a “V” shape was appeared at the center portion of the solution (i.e. an excellent stirred state).

The obtained particles were analyzed by X-ray diffraction (XRD), and were found to be wurtzite type zinc oxide. These zinc oxide particles were observed under a transmission electron microscope (TEM). TEM photographs are shown in FIGS. 1A and 1B. SEM photographs are shown in FIGS. 1C and 1D. As a result, it was found that the star-shaped zinc oxide particles having an average particle diameter of 1.3 μm were obtained.

### Example 1

### Preparation of Foundation

In accordance with the following formulation and production method, an O/W type of a liquid foundation was prepared.

### <Formulation A>

| | |
|---|---|
| Stearic acid | 4.0 parts by mass |
| Isotridecyl nonanoate | 2.0 parts by mass |
| Polyoxyethylene (10.E.O) cetyl ether | 0.3 parts by mass |
| Cholesterol | 0.2 parts by mass |
| Octyldodecyl oleate | 5.0 parts by mass |
| Behenyl alcohol | 0.5 parts by mass |
| Self-emulsifying propylene glycol stearate | 0.5 parts by mass |
| Methylpolysiloxane (20 cs) | 0.5 parts by mass |

### <Formulation B>

| | |
|---|---|
| Sorbitol solution | 2.0 parts by mass |
| Dipropylene glycol | 3.0 parts by mass |
| Sodium cetyl sulfate | 0.1 parts by mass |
| Triethanol amine | 0.8 parts by mass |
| Aluminum magnesium silicate | 0.05 parts by mass |
| Purified water | Balance |
| Methyl p-oxybenzoate | Optimized |

### <Formulation C>

| | |
|---|---|
| Star-shaped zinc oxide particles of | 5.0 parts by mass |
| Synthesis Example 1 | |
| Iron red | 0.3 parts by mass |
| Yellow iron oxide | 0.5 parts by mass |
| Black iron oxide | 0.2 parts by mass |
| Talc | 1.0 part by mass |

### <Formulation D>

| | |
|---|---|
| 2% by mass xanthan gum solution | 10.0 parts by mass |
| Total: | 100.0 parts by mass |

### <Production Method>

The ingredients of the formulation C were mixed and pulverized in advance, and then were added to the formulation B whose ingredients were heated and melted at 80° C. Then, this mixture was added to the formulation A whose ingredients were heated and melted at 80° C. in advance, and the mixture was emulsified by an emulsifying device (a homogenizer). The temperature of the mixture was dropped to 40° C. while stirring, then the formulation D was added thereto. The temperature thereof was cooled down to room temperature to thereby obtain a liquid foundation.

### Example 2

### Preparation of Foundation

A foundation was prepared in the same manner as in Example 1, provided that the formulated amount of the star-shaped zinc oxide particles of Synthesis Example 1 was changed to 10.0 parts by mass.

### Example 3

### Preparation of Foundation

A foundation was prepared in the same manner as in Example 1, provided that the formulated amount of the star-shaped zinc oxide particles of Synthesis Example 1 was changed to 2.0 parts by mass.

### Comparative Example 1

### Preparation of Foundation

A foundation was prepared in the same manner as in Example 1, provided that the star-shaped zinc oxide particles of Synthesis Example 1 were replaced with titanium oxide particles (CR-50, manufactured by Ishihara Sangyo Kaisha, Ltd., polygon, average particle diameter of 0.25 μm). Note that, the titanium oxide particles were those surface-treated with octyltriethoxysilane.

### Comparative Example 2

### Preparation of Foundation

A foundation was prepared in the same manner as in Example 1, provided that the star-shaped zinc oxide particles of Synthesis Example 1 were replaced with commercially available zinc oxide particles (NONO BYK-3840, manufactured by BYK Japan K.K., irregular shapes, average particle diameter of 0.04 μm, see FIG. 2). Note that, as these zinc oxide particles were dispersed in water, they were dried once, then surface-treated with octyltriethoxysilane.

### Comparative Example 3

### Preparation of Foundation

A foundation was prepared in the same manner as in Example 1, provided that the star-shaped zinc oxide particles of Synthesis Example 1 were replaced with zinc oxide particles (sphere) prepared in the following manner.

—Preparation of Zinc Oxide Particles (sphere)—

Using zinc sulfate heptahydrate (manufactured by Kanto Chemical Co., Inc., purity of 99.5%), a 0.2 M zinc sulfate (ZnSO₄) solution (pH of 5.6) was prepared, and a 4.0 M sodium hydroxide (NaOH) solution (pH of 13.8) was prepared using sodium hydroxide (manufactured by Junsei Chemical Co., Ltd., granular, purity of 99.9%). Into a mixed solution (the temperature thereof was maintained at 60° C.) of pure water (500 mL) and the ZnSO₄ solution (250 mL), 250 mL of the NaOH solution (pH of 13.8) was added at about 50 mL/min. The mixture was allowed to react at 60° C. for 2 hours, while stirring. After repeating a sufficient wash with water, a filtration was performed in the end, to thereby obtain a dry powder.

The obtained particles were analyzed by X-ray diffraction, and found to be wurtzite-type zinc oxide. It was also found as a result of the scanning electron microscopic (SEM) observation that the obtained particles were spherical particles having an average particle diameter of 0.05 μm. The aforementioned operations were repeated until an amount of the particles, which was sufficient for the evaluation, was obtained. Finally, the obtained particles were surface-treated with octyltriethoxysilane.

### Comparative Example 4

### Preparation of Foundation

A foundation was prepared in the same manner as in Example 1, provided that the star-shaped zinc oxide particles of Synthesis Example 1 were not added.

Next, the particles used in Examples 1 to 3 and Comparative Examples 1 to 3, and the foundations of Examples 1 to 3 and Comparative Examples 1 to 4 were evaluated in terms of ultraviolet-ray absorption, wide-angle scattering, transparency, and feelings (adhered feelings) at the time when applied, as follows. The results are shown in Table 1.

### <Diffuse Reflectance Spectrum (Ultraviolet Ray Absorption)>

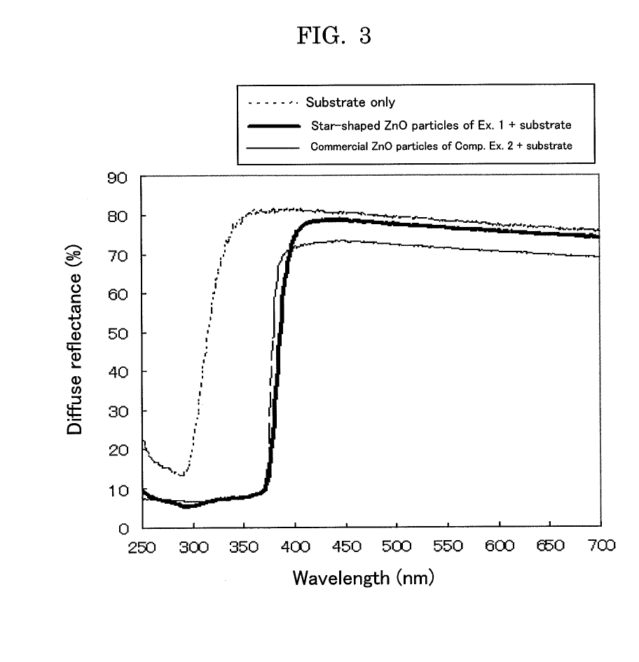

The star-shaped zinc oxide particles of Synthesis Example 1 used in Example 1 were dispersed in water, and the dispersion was applied to a glass substrate (borosilicate glass having polished end faces No. 2 S9112, a large glass microscopic slide, manufactured by Matsunami Glass Ind. Ltd.,), and then dried, to thereby obtain a sample. A sample was also prepared in the same manner using the commercial zinc oxide particles (irregular shapes) used in Comparative Example 2. The obtained samples were subjected to the measurement of a diffuse reflectance spectrum (measured by HITACHI U-3310 connected with an integrating sphere-attached device). The results are shown in FIG. 3. From the results of FIG. 3, it was found that the star-shaped zinc oxide particles used in Example 1 had excellent ultraviolet absorbing properties.

### <Wide-Angle Scattering>

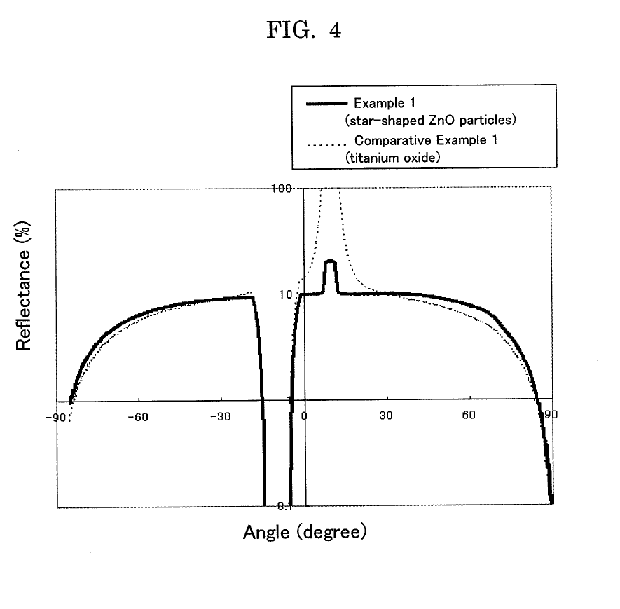

Each of the particles used in Examples 1 to 3 and Comparative Examples 1 to 3 were applied onto a glass plate having an adhesive sheet in the smallest amount, and were lightly spread by a finger. The particles were spread on the glass plate repeatedly until the layer of the particles could not be thinner, to thereby form a thin coated layer. These thin coated layers were subjected to the measurement by means of a goniometer device (a deflection spectral reflectance measuring device)(a goniophotometer GP-5, manufactured by Murakami Color Research Laboratory Co., Ltd.), and the results were evaluated in terms of wide-angle scattering properties based on the following criteria. The results of the star-shaped zinc oxide particles used in Example 1 and the titanium oxide particles used in Comparative Example 1 were shown in FIG. 4. In FIG. 4, the peak present adjacent to 10 degrees on a horizontal axis shows a peak corresponding to regular reflection, and the peak present adjacent to −10 degrees on the horizontal axis shows a peak corresponding to incident light. It was found that the reason why the intensity of Example 1 was low because the star-shaped zinc oxide particles had low regular reflection.

Next, each of the foundations of Examples 1 to 3 and Comparative Examples 1 to 4 was subjected to the measurement by means of the goniometer device in the same manner, and evaluated based on the following criteria.

### [Evaluation Criteria]

Taking the reflectance of Comparative Example 1 as a standard (B), the reflectance being in the range from −90° to −30° in the goniometer measurement result shown in FIG. 4 (horizontal axis: angle vs longitudinal axis), the results better than Comparative Example 1 were evaluated as A, the results similar to Comparative Example 1 were evaluated as B, and the results worse than Comparative Example 1 were evaluated as C.

### <Transparency>

Each of the particles used in Examples 1 to 3 and Comparative Examples 1 to 3 were applied onto a glass plate having an adhesive sheet in the smallest amount, and were lightly spread by a finger. The particles were spread on the glass plate repeatedly until the layer of the particles could not be thinner, to thereby form a thin coated layer. These thin coated layers were subjected to the measurement of a transmittance by means of a haze meter NDH-200, manufactured by Nippon Denshoku Industries Co., Ltd.

### [Evaluation Criteria]

A: The transmittance was 60% or more.
B: The transmittance was 40% or more, but less than 60%.
C: The transmittance was less than 40%.

### <UV-ray Absorption>

Each of the foundations used for the aforementioned goniometer measurement was subjected to the measurement of a diffuse reflectance spectrum (a measuring device: HITACHI U-3310 connected to an integrating sphere-attached device), and evaluated based on the following criteria.

### [Evaluation Criteria]

A: The spectrum showed ultraviolet-ray absorption
B: The spectrum did not show ultraviolet-ray absorption, or it was not clear on the spectrum.
<Feelings at the time of the Application (Adhered Feeling)>

Ten panelists applied each foundation on their faces after washing the faces, and then evaluated feelings or textures (adhered feeling) at the time when each foundation was applied onto their faces based on the following criteria.

### [Evaluation Criteria]

A: Eight out of 10 people felt it as excellent.
B: Six to seven out of 10 people felt it as excellent.
C: Four to five out of 10 people felt it as excellent.
D: Fewer than 4 out of 10 people felt it as excellent.

TABLE 1

| | | | | | Com. | Comp. | Comp. | Comp. |
|---|---|---|---|---|---|---|---|---|
| | | Ex. 1 | Ex. 2 | Ex. 3 | Ex. 1 | Ex. 2 | Ex. 3 | Ex. 4 |
| UV-ray shielding | Type | ZnO | ZnO | ZnO | TiO₂ | ZnO | ZnO | N/A |
| agent | Shape | Star | Star | Star | Polygon | Irregular | Sphere | N/A |
| | Average particle | 1.3 μm | 1.3 μm | 1.3 μm | 0.25 μm | 0.04 μm | 0.05 μm | N/A |
| | diameter | | | | | | | |
| | Formulated | 5.0 | 10.0 | 2.0 | 5.0 | 5.0 | 5.0 | N/A |
| | amount (mass %) | | | | | | | |

| | | | | | | | |
|---|---|---|---|---|---|---|---|
| Wide-angle scattering | A | A | A | B | C | C | N/A |
| properties of uv-ray | | | | | | | |
| shielding agent per se | | | | | | | |
| Wide-angle scattering | A | A | B | B | C | C | C |
| properties of foundation | | | | | | | |
| Transparency of uv-ray | A | A | A | C (22%) | A (84%) | A (79%) | N/A |
| shielding agent per se | (72%) | (72%) | (72%) | | | | |
| (transmittance) | | | | | | | |
| UV-ray absorption | A | A | A | B′ | A | A | B |
| Feelings at the time of | A | A | A | A | C | B | D |
| the application | | | | | | | |
| (adhered feeling) | | | | | | | |

The cosmetic composition of the present invention has transparency, ultraviolet-ray shielding properties for UV-A range (320 nm to 400 nm), wide-angle scattering properties, and excellent adhered feelings, and can be used, for example, as a toner, a moisturizer, a cream, ointment, a foundation, a lip balm, a lip stick, a mascara, an eye shadow, an eye brow pencil or marker, and a nail polish

What is claimed is:

1. A cosmetic composition, comprising:
star-shaped zinc oxide particles each having a star shape, and containing zinc oxide as a main substance thereof.

2. The cosmetic composition according to claim 1, wherein the star-shaped zinc oxide particles are formed by accumulating particles having a diameter of 1 nm to 100 nm so that crystallographic orientations thereof are aligned in the same direction, and allowing the particles to grow in a branched manner, and wherein the star-shaped zinc oxide particles have an average particle diameter of 1 μm to 3 μm.

3. The cosmetic composition according to claim 1, wherein an amount of the star-shaped zinc oxide particles is 0.1% by mass to 90% by mass with respect to the total amount of the cosmetic composition.

4. The cosmetic composition according to claim 1, wherein the star-shaped zinc oxide particles are subjected to a surface treatment with a surface-treating agent.

5. The cosmetic composition according to claim 4, wherein the surface-treating agent is a silane-coupling agent.

6. The cosmetic composition according to claim 1, wherein the cosmetic composition is a foundation.
